(19) United States
(12) Patent Application Publication
(10) Pub. No.: US 2020/0272761 A1
(43) Pub. Date: Aug. 27, 2020

(54) SOFTWARE HAVING CONTROL LOGIC FOR SECURE TRANSMISSION OF PERSONAL DATA VIA THE INTERNET FROM COMPUTERS TO THE SERVER, WITH SECURE STORAGE OF THE DATA ON SERVERS

(71) Applicant: Thomas Krech, Frauenfeld (CH)

(72) Inventor: Thomas Krech, Frauenfeld (CH)

(21) Appl. No.: 16/756,817

(22) PCT Filed: Mar. 21, 2017
(86) PCT No.: PCT/CH2017/000030

§ 371 Date: Apr. 16, 2020

(30) Foreign Application Priority Data
Mar. 21, 2016 (CH) 00389/16

(51) Int. Cl. G06F 21/62; G06N 5/04; G16H 10/60

(52) U.S. Cl. CPC G06F 21/6254; G16H 10/60; G06N 5/04

(57) ABSTRACT

The invention relates to a personal data network having a server device for storing personal data of a user, and having a computer unit associated with a user. The computer unit and the server device communicate via a network in order to exchange de-personalized data. The personal data network arises from the fact that only data which permit no direct or indirect conclusions to be drawn about the person is conducted via the network and stored in the network. Of The personal data of the user is already de-personalized on the computer unit by an identifier and is transmitted to the server device, where it is stored in a de-personalized manner under the identifier. The identifier results from an allocation rule stored only on the computer unit. Personalization of the de-personalized personal data in the form of an allocation of the de-personalized personal data to the associated user is not possible

### CROSS-REFERENCE TO RELATED APPLICATIONS

This application is a national phase entry under 35 U.S.C. § 371 of PCT/CH2017/000030 filed Mar. 21, 2017, which claims priority to Swiss Patent Application No. 00389/16 filed Mar. 21, 2016, the entirety of each of which is incorporated by this reference.

### TECHNICAL FIELD OF THE INVENTION

The present invention relates to methods by means of which the security of personal data in electronic networks and on servers can be increased. This relates, in particular, to the acquisition, de-personalization, re-personalization, processing and modification of data of all kinds of a user, such as physician reports, diagnostic findings, vital data, data related to bank transactions, and government and private institutions of all types.

### PRIOR ART

A person's electronic data is usually distributed among a number of storage locations, starting with personal data, at the general practitioner, in hospitals, at health insurance companies, on smart phones—from fitness trackers, and vital data, as well as data from devices within the meaning of the Internet of Things, such as water and electricity meters—insurance data, all the way to bank account balances and data with Internet service providers, such as Google and Facebook. There is an increasing interest in merging this data, and in evaluating and making it available for the benefit of the user and the general public. The data of a user is often provided to persons or institutions in personalized form and retained by these, without this being necessary.

A growing interest exists among the users as well as on the part of legislators to de-personalize the personal data so as to avoid misuse of the personal data. The significance of de-personalizing the personal data increases when not only individual personal data is available, but all personal data of a user is merged, and such consolidated personal data is possibly stored centrally.

The idea of collecting personal data for the purpose of providing a patient-specific mobile record is being increasingly nurtured, especially in the USA, and is reflected, for example, in the iPhone application Health Chron (cf., https://www.linkedin.com/pulse/why-you-should-own-your-health-data-loc-pham).

A data collection platform is under development in the USA under the “PhysIQ” designation (http://www.physiq.com/markets/). This data collection platform continuously collects a user's vital data and makes it available to physicians, so as to allow them to respond proactively based on the vital data. The company developing the data collection platform formed a partnership with Samsung so as to utilize a data exchange platform developed by Samsung with the designation “SAMI” for transmitting sensor data to the cloud (cf., https://developer.samsungsami.io/sami/sami-documentation/).

Almost all patient records are already managed electronically in countries such as Denmark or the Netherlands. In contrast, the process of introducing a digitization of patient data is progressing only slowly in Switzerland or Germany (http://www.healthbytes.de/eu-studie-ehealth-durchdringung-allgemeinÄrzte-deutschland-potenzial/), for example. The EU launched the pilot study ‘epSOS’ (http://www.epsos.eu/home/download-area/information-on-healthcare-and-ehealth.html) with the goal of testing cross-border health services for EU citizens based on electronically stored personal information. In Switzerland, an interdisciplinary working group, IPAG EDP, within eHealth-Suisse is tasked with structuring electronic patient records (http://www.saez.ch/aktuelle-ausgabe/details/ipag-epd-nach-der-etappe-ist-vor-der-etappe.html). In Switzerland, the legal aspects are set out in the Federal Act on the Electronic Patient Dossier (EPDG).

It is the object of EP-A-1 939 785 to purge patient data of personally identifiable information, so as to allow research institutes, for example, to evaluate the de-personalized data stemming from a large number of patients and from different sources created at different times. It is another objective of EP-A-1 939 785 to ensure that the de-personalized patient data for a particular patient can be correlated, without having to resort to the personal data. This is achieved by providing patient data, stemming from a person, with an anonymous, encrypted linking code by means of a program installed on the processor. To this end, the personal data that allows the person to be inferred is deleted in the original patient data before the data is forwarded to a database. According to EP-A-1 939 785, there is a functional relationship between the anonymous linking code and the personal data, so that each data source is able to create the anonymous linking code from the same predetermined portions of the personal data. The predetermined portions of the personal data are first (separately) encrypted, and then input into a first hash function. The output from the first hash function is input into a second hash function together with the anonymous linking code. The output of the second hash function is then encrypted before the data is transmitted to a server. The data is decrypted on the server and is then available for patient-related and other evaluations, without requiring knowledge of the personal data.

WO 01/18631 (D1), in turn, describes a method in which de-personalized patient data is stored on a server and provided to users (patient or physician). The data is provided with an identifier, from which a hash value is generated, which optionally can also be encrypted.

The US patent application no. 2006/0179073 (D3) describes an information management device for processing data containing personal data, in which the personal data is extracted from the processing object data, and a unique code is generated based on this data. The personal data of the processing object data is subsequently replaced with the unique code so as to generate primary conversion data. During a transmission of the primary conversion data, the data is additionally encrypted with a predetermined password. The described method preserves the value of the original data for statistical evaluations, even if the personal data has been replaced with the unique code.

### ADVANTAGES OF THE INVENTION

The invention set forth herein provides a personal data network (1, 28) and software having a control logic for the use in a personal data network (1, 28), which is improved with respect to
the security of the personal data in terms of unauthorized access by third parties;
the de-personalization of the personal data of one user and/or of a plurality of users, and/or
the generation of personalized personal data from the de-personalized personal data.

In addition, the personal data network is able to manage personal vital data of a user. The user is also notified about potential health problems as early as possible.

### SOLUTION

According to the invention, the advantages of the invention are achieved by the features of the independent claims. Further configurations according to the invention are apparent from the dependent claims.

### SUMMARY OF THE INVENTION

A server device is used in a personal data network according to the invention. This server device can be a single server unit or multiple server units networked with one another. Personal data of a user is stored on the server device. Such personal data includes, for example, vital data of a user, such as blood pressure, body temperature, activity information, such as a distance covered, or a number of steps walked or flights of stairs climbed, and the like, wherein these vital data can be derived, for example, from what are known as wearables, such as wristbands (http://www.aerzteblatt.de/nachrichten/62732), a smart phone fitness application (such as that sold, for example, under the “Endomondo” designation, or such as the HealthKit from Apple, or S Health from Samsung), building service engineering, or personal commodities, such as an electric toothbrush, a car, or mobile diagnostic devices (http://www.aerzteblatt.de/nachrichten/62729). Alternatively or cumulatively, it is possible for the personal data to be patient data, such as medical and treatment histories, physician reports, referral reports, examination findings such as X-ray images, ECG, laboratory findings, histological findings, images and videos of patients (in particular regarding results of examinations, skin changes, mucous membrane changes, wounds), data for the diagnosis and evaluation of the healing process, data acquired from hospital wards and intensive care units, genetic data, and the like.

The personal data network according to the invention furthermore has a computer unit, which is associated with a user. To mention a few non-limiting examples in this regard, the computer unit associated with the user can be a computer, a tablet, a cell phone, a smart phone, a smart watch, a wearable or a PDA.

Within the scope of the invention, the computer unit and the server device communicate via a network so as to enable an exchange of the personal data. This can take place, on the one hand, so as to provide the user with access to his or her personal data via the computer unit, and, on the other hand, so as to allow personal data to be transmitted from the computer unit to the server device for the purpose of being stored on the server device.

According to the invention, it is proposed that the personal data is not stored in the form of personalized personal data on the server device. In this way, third parties accessing the server device (with or without authorization) are precluded from gaining knowledge of the personalized personal data. According to the invention, it is proposed that the personal data is exclusively stored in the form of de-personalized personal data on the server device. Here, “de-personalized personal data” shall be understood to mean any form of the personal data that makes an allocation between the personal data and the person to whom this personal data relates impossible. To mention just a few non-limiting examples in this regard, the data cannot contain any designation, categorization or kind of header that includes a name of the person or another allocation to the person discernible for third parties. Alternatively or additionally, it is possible for all personal information to be removed from the personal data itself (for example, in a physician report or in another document, an image or the like).

However, without further measures according to the invention, the user would not be able to access the personal data since it would no longer be possible to locate the de-personalized personal data and associate it with the user, whereby the de-personalized personal data would be “lost.” To avoid this, an allocation rule is present on the computer unit associated with the user within the scope of the invention. The allocation rule can be arbitrarily designed per se, as long as it assigns an identifier to the user and his or her personal data, which per se does not allow third parties to discern the user described by this identifier. Just to mention a simple example that does not limit the invention, the identifier can be a simple number, which is individually assigned to the user. When the personal data is labeled with this number, the user, or the computer unit associated with the user, having knowledge of the identifier in the form of the number as a result of the allocation rule, is able to label and recognize the personal data labeled with the number as his or hers.

Within the scope of the invention, the personal data is stored on the server device both with the de-personalized personal data and with the identifier mentioned. Since it is not possible to personalize the de-personalized personal data in the form of an allocation of the de-personalized personal data with the associated user, based on the de-personalized personal data present on the server device and the identifier, the need to keep the personal data confidential and to protect the data against misuse is taken into account.

The invention proposes for the computer unit associated with the user to have a control logic, and in particular software, by means of which a request is generated to the server device that a transmission of the de-personalized personal data of this user is to take place. This request includes the identifier associated with the user. When the server device receives such a request including the identifier, the server device can select, from a variety of de-personalized personal data, the de-personalized personal data which is associated with the identifier, and to transmit this data to the requesting computer unit, whereby ultimately the de-personalized personal data associated with this user is transmitted to the user, and this data can be provided to the user for perusal.

So as to enable the transmission and the storage of a multitude of de-personalized personal data with the identifier, without the possibility of the associated person being inferred at the same time, the invention furthermore proposes to convert personalized personal data on the computer unit of the user into de-personalized personal data, using the allocation rule. By way of the control logic of the computer unit, the de-personalized personal data, with the associated identifier, is then transmitted from the computer unit to the server device via the network, where the data can then be stored without the possibility of the user being inferred. According to the invention, the personal data is thus only present in personalized form on the computer unit, but not on other parts of the personal data network. It is possible to delete this personal data after transmission on the computer unit.

It is possible that, in addition, authentication of the user and/or the computer unit is necessary, before communication and a transmission of the de-personalized personal data via the personal data network take place. The authentication can also be associated with the de-personalized personal data associated with the user in the process, so that the de-personalized personal data is only transmitted from the server device to the computer unit when, cumulatively, a request including the identifier associated with the de-personalized personal data is made, and the specific authentication for this de-personalized personal data is available. So as to increase the security, it is also possible that the key of a further person is necessary for authentication. This is also the case, for example, when a third person is to be granted access to the data or portions of the data of the user.

Another aspect of the invention relates to the problem that it is not just the identifier, a file name, a header or the like that allows the associated user to be inferred. Rather, the data of the aforementioned kind itself usually includes personal information. For example, the data may include the last name and/or first name of the user, date information, location information, zip codes, names of hospitals visited, names of health care facilities visited, accident sites, telephone numbers, signatures and other person-specific information. In another configuration of the invention, it is proposed that the computer unit associated with the user has a control logic, by means of which it is possible to remove personal information and information allowing the person to be inferred from information unit data encompassed by the personalized basic data, or to convert it. It is possible in the process for the “personalized personal data” to be a comprehensive health record, while the information unit data relates to individual “sheets” or portions of this personal data record. Just to mention a few non-limiting examples, the information unit data can be a picture (which shall be understood to also include an image sequence in the form of a video), a text, an audio file (for example, including a dictated physician report, an echocardiogram etc.), and the like. It is possible for the personal information to be removed manually by the user. Alternatively or cumulatively, it is possible that the personal information is removed automatically. Instead of removing the personal information, it is likewise possible to convert the personal information such that partial information is still present, which then, however, no longer allows the user to be inferred, or only to a reduced degree.

A variety of options exist for automatically identifying the personal information for the purpose of removal or conversion. For one suggestion of the invention, the computer unit of the user has a control logic, which includes a recognition logic. Personal information and information allowing the person to be inferred can be automatically recognized in the information unit data by means of the recognition logic. For example, the recognition logic can include OCR recognition, which converts a graphic included in the information unit data into text. Using logical conditions and known formation laws, this text can then be searched for personal information and information allowing the person to be inferred, for example
by searching for date information in a predefined date format, or
by searching for text components corresponding to names, wherein it is also possible to resort to a relevant database with respect to the names to be considered, and the like.

When the recognition logic leads to detected personal information, the detected personal information can be removed directly from the information unit data. In the event that the recognition is based on OCR recognition, the portion of the image correlating with the text component according to the OCR recognition has to be permanently removed for this purpose. It is also possible for a query to be sent to the user prior to removing the personal information from the information unit data, who has to provide a confirmation prior to the removal.

For a personal data network according to the invention, the computer unit of the user has a control logic, which converts at least a portion of the recognized personal information in the information unit data into generalized personal information and information allowing the person to be inferred. Just to mention one non-limiting example in this regard, personal information may consist of date information including the day, month and year. In this case, the date information can be converted into generalized personal information that only includes the year. It would also be possible, for example, for the personal information to include a place of residence or a state, while a conversion into generalized personal information takes place in the form of a state or a country or a larger territory. The generalized personal information ensures the need for confidentiality with respect to the personal data. On the other hand, for example, it is possible to analyze the personal data, such as the examination of a development of a disease over the course of many years, despite the elimination of the day and the month, within the scope of the conversion, or it is possible to carry out statistical examinations with respect to disease incidences, taking special regional circumstances into consideration, based on the generalized personal information in the form of the state, country or territory. Another option would be for a control logic to convert the intervals between events identified by date into time intervals. This would be, for example, “onset of disease on such and such date in such and such year, and hospitalization on such and such date in such and such year” converted into “hospitalization 10 days after onset of disease.”

For another suggestion according to the invention, the recognition logic includes a text, image or audio recognition logic. By way of a comparison of recognized words, images or audio components to predetermined words, images, audio components, or formation laws, the control logic identifies personal information and information allowing the person to be inferred, for which purpose it is also possible to utilize corresponding databases including possible personal information. The control logic then removes the personal information thus identified from the information unit data, or converts this information (for example, as mentioned above) into generalized personal information.

If the user is to be involved in the conversion or removal of personal information, this can take place for another suggestion of the invention in that the control logic of the computer unit of the user outputs information unit data on an output of the computer unit, and in particular a screen or a speaker. The control logic then makes it possible for the user to remove personal information and information allowing the person to be inferred, identified by the user in the information unit data based on the output. Just to mention one non-limiting example, the user can mark a portion of the output information on a screen, which is then converted or removed. The user can carry this out based on the visual inspection of the output. It is also possible that, based on an automated process, different identified personal information and information allowing the person to be inferred, along with a prompt to confirm whether and, if necessary, to what extent, personal information and information allowing the person to be inferred is to be converted or removed, are consecutively displayed to the user.

It is possible for converted or removed information in the personal data to be permanently lost. If, in contrast, it is to be made possible for (in particular only) the user to reconstruct the full personal data, including the converted or removed information, at a later point in time, the invention proposes to store personal information and information allowing the person to be inferred, which was removed from the information unit data, or converted, by means of the control logic of the computer unit, which likewise may take place on the computer unit. Both the deleted or converted personal information itself, and the location at which the deleted or converted personal information was present in the information unit data or the personal data, may be stored in the process. For an at least partial reconstruction of the original personal data or information unit data, the control logic of the computer unit may add the stored personal information back to the de-personalized personal data which was received from the server device via the network and in which personal information and information allowing the person to be inferred was removed from information unit data, whereby an at least partial completion and/or restoration takes place.

It is certainly possible that exclusively several computer units, each associated with a user, communicate with the server device in the personal data network. The larger the number of users and of the associated computer units, the less is it possible to associate the de-personalized personal data with the users and computer units. So as to enable, in particular when starting to populate the server device for the transmission of data of the first users, an association with these users, the server device can, at least initially, also be populated with de-personalized personal data of fictitious users created, for example, based on random criteria.

However, it is also possible within the scope of the invention for additional devices to be integrated in the personal data network:

For one suggestion of the invention, supporter computer devices, in particular computer units of computer sub-networks, are integrated into the personal data network, which are associated with supporters for the users. Such supporters may be health care staff, such as the physician or a treating person or a caregiver, a practice or a hospital. Other supporters who may be integrated into the personal data network by an associated supporter computer device include pharmacies, insurance companies, banks or research facilities, just to name a few.

If a supporter computer unit is to be enabled to receive de-personalized personal data from the central server device, this can take place in two different ways:
a) it is possible for the user himself or herself to transmit the personal data from the computer unit to the supporter computer unit using a different path, for example by way of a wireless or wired network;
b) it would also be possible, however, for the user to transmit the allocation rule or the identifier to the supporter computer unit, whereby the supporter computer unit is then able to access the server device by querying the de-personalized personal data that belongs to the user, or, in this case, the patient of the supporter.

It is also possible for the personal data network to have an analysis interface. Using the analysis interface, the personal data network can communicate with an analysis computer unit, in which personal data can be analyzed, for example for the diagnostic evaluation of the personal data of a user and/or for the collection or statistical examination of the personal data of multiple users.

The invention furthermore proposes for the supporter computer unit, the analysis computer unit, the server device and/or the computer unit associated with the user to have a control logic, which ascertains findings from the de-personalized personal data. The server device may have a data collection and a control logic, which makes it possible to ascertain findings from the de-personalized personal data of a user and to inform the user accordingly. The information can be sent either directly to the cell phone of the user or, for example, to an interposed server, wherein the latter then forwards the information to the user.

Alternatively or cumulatively, it is possible for the control logic to generate automatic messages. Just to mention a few non-limiting examples, a critical circulatory condition can be ascertained from vital data (such as the blood pressure and pulse), which were ascertained by way of a smart phone or a wearable. A generated automatic message can be, for example, an alert for the user or a companion of the user or a physician or other health care staff. It is also possible that a notice regarding a regular doctors visit (for example, about an impending vaccination after a predetermined time interval subsequent to the preceding vaccination) is generated as the automatic message and displayed on the computer unit of the user.

The invention also proposes for the supporter computer unit, the analysis computer unit, the server device and/or the computer unit associated with the user to have an acquisition device. Personalized or de-personalized personal data can be acquired by means of the acquisition device. It is also possible for the acquisition device to be a manual acquisition device, by means of which the user can enter personal data. This may be a keyboard. However, the acquisition device may be designed as a scanner, a camera, an audio recording device or the like, by means of which the personalized or de-personalized personal data may be acquired via the computer unit associated with the user.

It is likewise possible for an interface to be provided as an acquisition device on the computer unit, wherein the interface can have a wired or wireless design. This interface can be used to transmit, for example, personal data from a computer of an examination device of the physician or of the hospital, or to communicate with a scanner, a camera, an audio recording device or the like.

Alternatively or additionally, the computer unit associated with the user can have an interface to a vital data acquisition device, in particular a chest strap heart rate monitor, a wearable or the like. A “wearable” here shall be understood to mean a computer unit that is attached to the body of the user or his or her clothing during use. Alternatively, it is possible to receive already derived data via this interface, for example from the Apple® Health Kit (Apple Inc. brand) or Withings.

The problem with the personal data network may be that the personal data is transmitted in de-personalized form between the computer unit and the server device, so that it is not possible to directly associate the personal data with the user based thereon. However, under some circumstances it is possible to ascertain an IP address from which the computer unit has sent the de-personalized personal data, from the transmission path of the de-personalized personal data, whereby ultimately it would be possible to infer the user or at least an environment of the user. If this is to be avoided, a transmission path disconnection device is interposed in a further configuration of the personal data network according to the invention between the computer unit associated with the user and the server device, which receives the de-personalized personal data transmitted from the computer unit and then transmits the de-personalized personal data to the server device, eliminating references to the IP address from which the transmission path disconnection device received the de-personalized personal data. In this way, it may be made impossible to infer the transmission path from the computer unit from the de-personalized personal data on the server device, whereby the confidentiality interests of the user and the protection against access to the personal data by third parties can be further taken into account. An interruption in the transmission path can take place, for example, by way of a virtual network by establishing a VPN connection to an interposed server, and by using the IP address of the interposed server when communicating with the server device. Services are available in the market, for example TunnelBear VPN™ (trade name of TunnelBear Inc.), which provide programs for desktop computers, cell phones, tablet computers and the like, allowing users to hide their own IP address. When the user is connected to the service, the actual IP address of the user is not evident on the websites he or she visits.

Another embodiment of the invention relates to the registration of the user. According to one suggestion, the computer unit associated with the user has a control logic which sends a telephone number of the computer unit, in particular of the smart phone. The computer unit then receives a code, which allows the computer unit to be authenticated, from the unit in charge of the registration, in particular the server unit to which the telephone number was sent from the computer unit. For example, when designed as a smart phone, the computer unit can receive this code in the form of an SMS. According to the invention, it is only after the code for authenticating the computer unit has been received that the computer unit, by way of the control logic, records personal data about the user, which can be the name, date of birth, place of birth and the like, for example. This personal data about the user can then be stored in the computer unit, wherein the data is, in particular, not transmitted via the network.

In principle, it is possible that an allocation rule and the identifier associated with a user thereby do not change. The security of the personal data network may possibly be further increased when a new allocation rule with a new identifier is ascertained by the control logic of the computer unit, either regularly or in relation to particular events. Using the ascertained new identifier, it is then possible to transmit de-personalized personal data from the computer unit to the server device via the network. Under certain circumstances,
the de-personalized personal data is transmitted from the server device to the computer unit with the old identifier;
the new allocation rule and the new identifier are ascertained by the computer unit;
the de-personalized personal data with the old identifier is deleted on the server device; and
the de-personalized personal data with the new identifier is transmitted back to the server device.

The aforementioned events, for which a new allocation rule with a new identifier is ascertained by the control logic of the computer unit, may be, for example, any process of transmitting the de-personalized personal data with the (old) identifier from the server device to the computer unit and/or the support device. Alternatively or additionally, the event that is used can be that a transmission of the allocation rule to a support device has taken place.

It is also conceivable that the described method, by means of which the user has the only key to access his or her data, can be supplemented or replaced with alternative methods, such as rolling code devices, paper code or personal features, such as iris recognition, video sequences of eye blinks, and the like, or DNA sequences.

It may be desirable that solely the user has personalized access to his or her personal data via the computer unit. However, this may be problematic in the event the computer unit is lost, the user faints or becomes incapable of acting. It is possible, as a provision for such instances, that the allocation rule is transmitted by way of the control logic of the computer unit to a computer unit associated with a person of trust. The person of trust is, for example, a spouse, a person authorized to make a decision for the user in an emergency, or a security person or a person fulfilling a fiduciary function, via whom access to the personal data is to remain possible if the computer unit is lost. It can then be made possible for the computer unit associated with the person of trust to at least partially access the de-personalized personal data and convert it into personalized personal data.

In principle, the invention also encompasses embodiments in which all personal information is completely de-personalized in the personal data. However, this may make it more difficult to evaluate the information scientifically or stochastically in another manner, with the objective of obtaining important findings. For a specific configuration of the invention, the invention proposes that the de-personalized personal data, with respect to the user, also includes
the year of birth,
the gender of the user,
the ethnicity, and
the affiliation with the state or canton of the place of residence or the country of residence

This data can then be utilized for further evaluation of the personal data, for example by scientific analysis institutes, insurance companies and the like.

In addition to the personal data network, the invention also relates to software, which is equipped with a control logic suitable for the use and the creation of a personal data network according to any one of the preceding claims. For this purpose, the software includes in particular a control logic as claimed in the claims.

The present invention furthermore relates to a system comprising a personal data network according to any one of claims 1 to 19, and a vital data acquisition device. The vital signals acquisition device can comprise one or more sensors for acquiring vital data. Possible sensors are sensors for acquiring the heart rate, the blood pressure, the glucose level, cardiac pacemakers, fitness trackers, position sensors, acceleration sensors and the like.

Advantageous refinements of the invention will be apparent from the claims, the description and the drawings. The advantages of features and of combinations of multiple features mentioned in the description are purely by way of example and can, alternatively or cumulatively, take effect, without necessarily having to achieve the advantages of embodiments according to the invention. Without thereby altering the subject matter of the accompanying claims, the following applies with respect to the disclosure of the original application documents and the patent: additional features can be derived from the drawings, in particular the shown geometries and the relative dimensions of multiple components with respect to one another and the relative arrangement and operative connection thereof. The combination of features of different embodiments of the invention or of features of different claims deviating from the selected back references of the claims is likewise possible and is hereby suggested. This also applies to those features that are shown in separate drawings or mentioned in the description thereof. These features can also be combined with features of different claims. Likewise, it is possible to eliminate features for further embodiments of the invention which are cited in the claims.

The features mentioned in the claims and the description shall be understood to mean, with respect to the number thereof, that exactly this number or a greater number than the described number is present, without requiring explicit use of the adverb “at least.” As a result, when one element is mentioned, for example, this shall be understood to mean that exactly one element, two elements or more elements are present. These features can be supplemented with other features or be the only features of which the particular product is composed.

The reference numerals contained in the claims do not constitute any limitation of the scope of the subject matter protected by the claims. They only serve the purpose of making the claims easier to understand.

### BRIEF DESCRIPTION OF THE DRAWINGS

The invention will be explained and described hereafter based on exemplary embodiments shown in the figures.

FIGS. 1 to 4 show different exemplary configurations of the de-personalized transmission of sensitive data via a network, typically the Internet. This network can additionally be configured with technical measures so that closed A to B connections arise, for example in the form of a virtual private network (VPN) or by tunneling;

FIG. 5 shows a highly schematic illustration of method steps of a control logic for the transmission of de-personalized data, the control logic relating to a method for an initial registration of a user;

FIG. 6 shows a highly schematic illustration of method steps of a control logic for the transmission of de-personalized data, the control logic relating to a method for a transmission of de-personalized personal data to a server device;

FIG. 7 shows a highly schematic illustration of method steps of a control logic for the transmission of de-personalized data, the control logic relating to a method for the de-personalization of personal data from a server device.

### DETAILED DESCRIPTION OF THE INVENTION

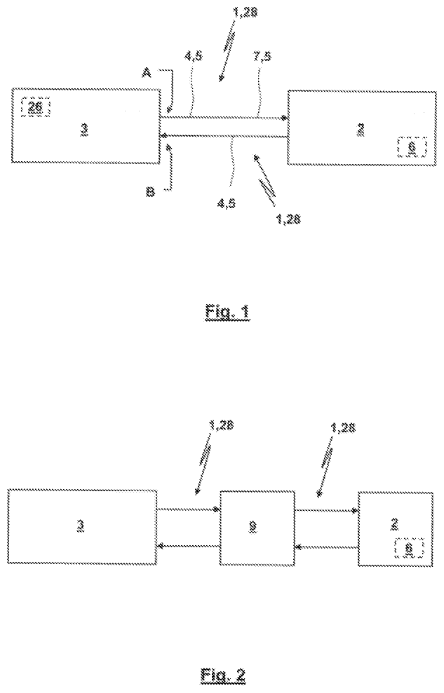

FIG. 1 shows a highly schematic illustration of a data network 1 having a server device 2 and one computer unit 3 associated with a user or patient, of a plurality of further computer units, not shown here, which communicate with server device 2 via a network 1, 28. Computer unit 3, on the one hand, transmits de-personalized personal data 4 with an associated identifier 5 to server device 2 so as to store de-personalized personal data 4 in a memory unit 6 of server device 2 under identifier 5. On the other hand, it is possible for computer unit 3 to transmit a request 7 to server device 2 with identifier 5, to transmit de-personalized personal data 4 associated with identifier 5 in the form of de-personalized personal data 4 to computer unit 3. The de-personalization is carried out by software A on computer unit 3, and the repersonalization is carried out by software B on computer unit 3.

So as to avoid that an association of stored de-personalized personal data 4 is already possible in server device 2 via a requesting IP address of computer unit 3, according to FIG. 2 a transmission path disconnection device 9 can be interposed between server device 2 and computer unit 3. Transmission path disconnection device 9 receives request 7 with identifier 5 from the IP address of computer unit 3, and transmits it with the own IP address, without reference to the IP address of computer unit 3, to server device 2. De-personalized personal data 4 associated with identifier 5 and stored in memory unit 6 is then transmitted from server device 2 to transmission path disconnection device 9, which then, in turn, transmits de-personalized personal data 4 to the IP address of computer unit 3 only available there.

With communication between computer unit 3 and server device 2 otherwise corresponding to FIG. 1, data network 1 according to FIG. 3 has a supporter computer unit 10, which is associated with a practice, a hospital, a bank or an insurance company, for example. So as to enable access to the personal data there, the user transmits de-personalized personal data 31 from computer unit 3 to supporter computer unit 10. Simultaneously or with time delay, identifier 5 is delivered, which includes the personal information. The personal information is inserted as a header into the document when the identifiers match. If further personal data is collected by the supporter, it is possible to re-transmit this personal data in the form of de-personalized personal data 4, having been provided with a randomly generated number (identifier 5a), to computer unit 3, by means of which it is then also possible to transmit this personal data in the form of de-personalized personal data 4 with associated identifier 5a for the purpose of additional storage in server device 2. Alternatively or additionally to supporter computer device 10, an analysis computer device 11 can have access to de-personalized personal data 33 of a plurality of users as a result of communication with server device 2. A plurality of de-personalized personal data 33 can then be analyzed in analysis computer device 11, and analysis result 32 of analysis computer device 11 can be transmitted to server device 2 or other devices. It is certainly also possible to transmit de-personalized personal data from supporter computer unit 10 to an analysis computer unit 11, whereby it is then possible for an analysis device to analyze this personal de-personalized data. Analysis computer device 11 communicates with an analysis interface 27 of server device 2 in the process. The result of the analysis can then be transmitted in de-personalized form to supporter computer device 10 or computer unit 3 of the user for further processing.

FIG. 4 shows an embodiment in which (alternatively or additionally) a supporter computer unit 10 or a person of trust computer unit 12 is integrated into data network 1, 28. So as to enable an exchange of the personal data associated with the user between server device 2 and supporter computer device 10 and/or person of trust computer unit 12, the user, by way of computer unit 3, transmits an allocation rule, in particular identifier 5 associated with the user, to supporter computer device 10 and/or to person of trust computer unit 12. Using this allocation rule and, if necessary, further transmitted authentications or passwords, data with respect to the de-personalized data associated with the user can then be exchanged between supporter computer device 10 and/or person of trust computer unit 12 on the one hand, and server device 2 on the other hand.

As an optional further possibility, FIG. 4 shows that computer unit 3 can also receive vital data 13 via an interface 29, which can originate from a wearable 14 or a vital data acquisition device 30, such as a wristband or a chest strap or an application of the smart phone forming computer unit 3.

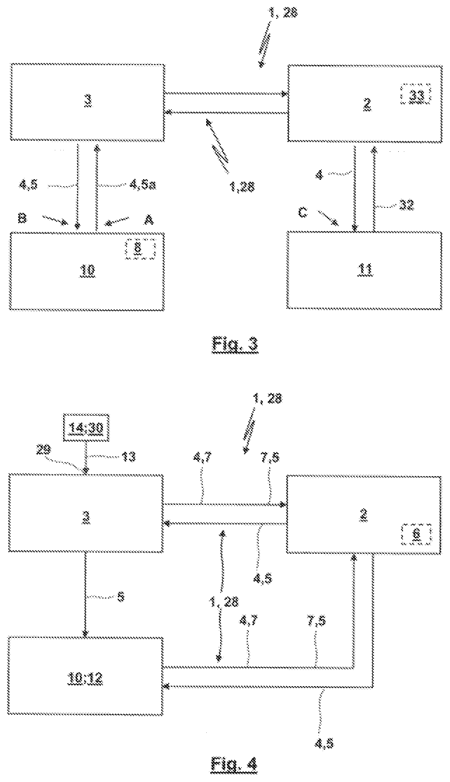

FIG. 5, by way of example, shows a method for an initial registration of a user by computer unit 3 associated with said user.

After loading an application onto computer unit 3 designed as a smart phone, for example, the user, in a method step 15, transmits his or her telephone number to the device carrying out the registration, in particular server device 2. However, it is also conceivable that a further server is connected upstream of server device 2 so as to strictly separate the registration data of the user from the data stored on server device 2. In a method step 16, identifier 5 is then ascertained, which is transmitted back to the smart phone in a method step 17, in particular by way of SMS, using the previously transmitted telephone number. Only then does the user enter the personal data in a method step 18. As a result of this registration, the smart phone or computer unit 3 is then operable. In particular, de-personalized data can then be transmitted with identifier 5 or 5a to server device 2 and/or the de-personalized data associated with the user can be loaded from server device 2 with reference to identifier 5 or 5a. Neither the personal data nor the association between the telephone number and the identifier is stored on server device 2. A corresponding procedure can be applied to the registration if computer unit 3 is not designed as a smart phone, but as a desktop version, for example, onto which the application can be loaded.

In a further method step, a query can optionally be sent to computer unit 3 as to whether a further computer unit 3 is to be registered as being authorized with respect to the user. In this case, the further telephone number, which is associated, for example, with a further smart phone, is then transmitted from computer unit 3 to server device 2.

The personal information to be entered in method step 18 is, for example, the last name, the first name, the date of birth, the gender, the ethnicity, the weight, the height, the street, the place of residence, the country, the e-mail address, the cell phone number, an identification card number or the like.

Under some circumstances, it can be checked in an additional method step whether the person logging in is a natural person. Moreover, the personal information can be verified through access to a corresponding database. It is also possible that consent with respect to the general terms and conditions is required during registration. The general terms and conditions can also include that the user agrees to the de-personalized data of the patient being scientifically evaluated and/or used financially.

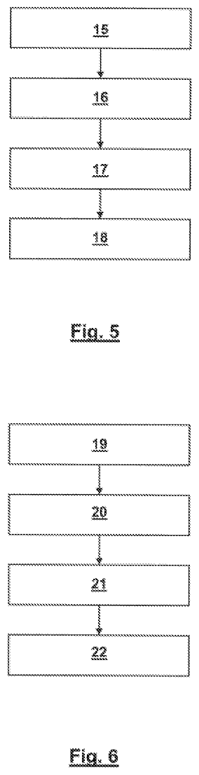

FIG. 6 shows the transmission of de-personalized personal data to server device 2 for the purpose of storing the same:

The initially described method steps are carried out either by computer unit 3, which can communicate with server device 2 (FIG. 1), or supporter computer device 10 or person of trust computer unit 12, which can communicate with server device 2 (FIG. 4).

In a method step 19, initially personal data is obtained. This can take place by receiving the personal data from an examination device, from supporter computer device 10 or by the acquisition of a physician report or the like, using an acquisition device 26. In a subsequent method step 20, personal information and information allowing the person to be inferred is then removed from information unit data, that is, the physician report, the X-ray image and the like, for example. Thereafter, in method step 21, the data packet, which includes both the de-personalized data and the associated identifier, is transmitted to server device 2. In method step 22, server device 2 then stores the received de-personalized data under identifier 5 in memory unit 6.

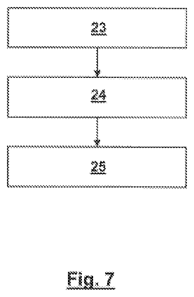

FIG. 7 shows the method for loading de-personalized data from server device 2 into computer unit 3 (FIG. 1) or into supporter computer device 10 or person of trust computer unit 12 (cf., FIG. 4).

In a method step 23, computer unit 3 (or supporter computer device 10 or person of trust computer unit 12) transmits a request 7 with identifier 5 to server device 2. In a method step 24, server device 2 loads the de-personalized data associated with identifier 5 from memory unit 6. The de-personalized data is then transmitted in method step 25 to computer unit 3 (or supporter computer unit 10 or person of trust computer unit 12), where it is re-personalized, for example in that the personal data is listed in a header.

Server device 2 can initially store the de-personalized data of many users on memory unit 6 according to respective identifier 5. This storage record encompassing data from multiple users is shown in the figures as reference numeral 33. It is likewise possible to extract search terms to enable a search function for analyzing the de-personalized data of multiple users (33). The de-personalized data with respect to a user can be categorized according to the type of information unit data, for example. In this way, it is possible, for example, to carry out a classification between examination findings, physician letters, discharge reports. It is likewise possible that the information unit data or identifier thereof is summarized as a function of the institute carrying out the individual examinations and physician reports. Alternatively or additionally, it is possible to categorize the information unit data as a function of disease or affected body part or the medical specialty. It is possible to provide information unit data of different categories individually to the user or a supporter.

An application of computer unit 3 can include a search function so as to allow information to be retrieved easily.

Examples of wearables 14 that can be used in the data network 1 within the scope of the invention are listed, for example, on the website (http://www.emdt.co.uk/daily-buzz/5-wearables-could-transform-healthcare). Examples include:
“Google lens” for glucose monitoring;
“WearSens” for measuring food intake (cf., http://www.medicaldaily.com/ucla-engineers-develop-wearsens-food-diary-you-can-wear-around-your-neck-324508);
Google smart pill for detecting cancer (cf., http://mobihealthnews.com/37730/google-x-developing-cancer-scanning-pill-that-transmits-to-a-wearable-sensor/),
wearable sensors for continuously measuring body fluids (cf., http://www.emdt.co.uk/daily-buzz/higher-powered-wearable-sensors), and
sensors for continuous ECG measurement (cf., http://internetmedicine.com/irhythm/).

An analysis device integrated into data network 1 can evaluate a plurality of de-personalized data of one patient or of multiple patients, wherein an additional validation and release from a medical perspective can take place. The results from the analysis or the generated data extracts can, in particular, include the following information and data:
an electronic physician letter (cf., http://www.aerzteblatt.de/archiv/167716/Elektronischer-Arztbrief-Arztnetze-fuer-die-Erprobung-gesucht),
discharge reports, bank statements;
diagnostic lists, expense lists according to category as bank account statements;
referral reports, payment transfers, damage reports;
risk analyses;
summaries on particular problems;
reminders and prompts for doctor's visits, for vaccination, for cancer screening, for taking drugs and the like, for standing orders for bank transactions;
evaluations and assessments of medical images (X-ray images, hemograms, skin changes);
alerts for health care staff in the case of critical vital data of patients in the intensive care unit, in the ward, in the nursing home or in independent assisted living; and
evaluations for the pharmaceutical industry, for insurance companies, and for scientific purposes.

Computer unit 3, in particular by way of a smart phone application, is used for registering, temporarily storing the personal data, procuring and inputting new personal data, de-personalizing the personal data, generating identifiers for use with de-personalized data packets, and establishing the connection to server device 2, along with the subsequent upload.

In contrast, server device 2 is used for managing the de-personalized personal data with the associated identifier, and managing access for the users as well as analysis devices and the like.

An application of a computer device 3 designed as a smart phone can include, for example, a menu interface, which includes the following menu items:
login to allow login using a user name and a password
“my personal data online”;
“my next appointments”;
“scan personal data”;
“import personal data”;
“process personal data”;
“request prescription”;
“conduct lab analysis”;
“'schedule doctor's appointment”;
“chat”,
“settings” and/or
“manage favorites”
   and can execute the associated functions.

A transmission of de-personalized data via data network 1, 28 is carried out, in particular, in the form of metadata according to IHE standard. It is possible, for the re-personalization, to insert identifier 5 as a header into the document, instead of (re-)inserting the patient data into the document, the header being linkable by the user to his or her personal data using the allocation rule. The de-personalized data is then downloaded and uploaded via an encrypted connection. The de-personalized personal data can additionally be encrypted according to the customary known encryption technologies.

It is possible to manage access for third parties, such as persons of trust or analysis devices, by way of a protocol with the “OAuth” identifier, as is described in the relevant technical literature and in https://en.wikipedia.org/wiki/OAuth. Based on saved profiles, the user can individually determine and control, by way of the application on computer unit 3 or server device 2, the components of the de-personalized data that third parties are able to access.

It is possible that the user, when creating his or her profile, has to consent to a trustee forwarding the personal data to third parties, authorized agents or a person of trust, possibly of last resort. Data access by a representative is considered, in particular, when the user is unconscious, under guardianship, deceased or when computer unit 3, which exclusively contains the allocation rule, has been lost.

It is possible that a method described on the website https://validic.com/api is used within the scope of the invention.

So as to eliminate personal information, a document, such as a physician letter or an X-ray image, can initially be completely redacted or deleted, whereupon the user can then “selectively” reactivate individual components of this document using a kind of swipe function. Conversely, the user can personally redact passages in his or her document that have not been redacted yet and that allow his or her identity to be inferred, by using a swipe function.

### EXEMPLARY FUNCTION SEQUENCE

The user uses his or her smart phone to directly forward his or her vital data from a fitness tracker, a blood pressure meter or a cardiac pacemaker to the smart phone, for example via Bluetooth. It is also conceivable that the data has already been transmitted to different software, such as the Apple Health Kit. In the latter case, the data from such a system is transferred to the smart phone. Ultimately, it is also conceivable that the smart phone is used to scan medical finding texts and images using a special function. In all cases, the data is de-personalized by way of automatic programs. Where this is not possible, personalized data can be removed by way of a swipe function. It is of importance now that the entire de-personalization process takes place on the smart phone. Only thereafter the data is transmitted in de-personalized form to the server. Here, a continuous aggregation of the data and analysis of the data owner take place, and feedback is output on the smart phone of the data owner in the event of deviations.

The invention relates to a personal data network 1 comprising a server device 2 for storing personal data of a user, and a computer unit 3 associated with a user, in particular a smart phone, a tablet PC or an iPad and a desktop PC. Computer unit 3 and server device 2 communicate via a network 1, 28 so as to exchange de-personalized data.

The personal data network is created in that only data that does not allow the person to be inferred, directly or indirectly, is routed via the network and stored in the network; this is so-called “de-personalized” data.

According to the invention, the personal data of the user is already de-personalized with an identifier 5 on computer unit 3 and transmitted to server device (2), where it is stored in de-personalized form under identifier 5. Identifier 5 results from an allocation rule, which is stored exclusively on computer unit 3. It is not possible to personalize de-personalized personal data 4 in the form of an association of the de-personalized personal data with the associated user based on de-personalized personal data 4 and identifier 5 present on server device 2. Furthermore, an association during the transmission of the de-personalized data via the network is likewise not possible.

In the form of an alias identity, the identity of the user can be “loaned” in the form of an identifier 7 on a temporally limited or permanent basis, for example to a supporter computer device 10 or a trustee computer unit 12, so that data incurred there can be transmitted in de-personalized form under loaned identifier 7 to the server unit.

1-21. (canceled)

22. A data network, comprising:
a server device, on which personal, sensitive data of a user is stored; and
a computer unit associated with a user, the computer unit and the server device communicating with one another via a network so as to exchange the personal data;
an allocation rule being present on the computer unit associated with the user, which allocates an identifier to the user and his or her de-personalized personal data, the personal data being exclusively stored on the server device in the form of de-personalized personal data with the identifier, a personalization of the de-personalized personal data in the form of an allocation of the de-personalized personal data to the associated user, based on the de-personalized personal data and the identifier present on the server device, not being possible;
the computer unit associated with the user comprising a control logic, by which:
a request is generated to the server device with respect to the transmission of the de-personalized personal data, the request including the identifier associated with the user, and the de-personalized personal data, which is transmitted in response to the request from the server device to the computer unit via the network and with which the identifier is associated, being received; or
personalized personal data on the computer unit is converted into de-personalized personal data with the associated identifier, using the allocation rule, and the de-personalized personal data, with the associated identifier, is then transmitted from the computer unit via the network to the server device;
the computer unit associated with the user having an interface to a vital data acquisition device; and
the computer unit associated with the user having the control logic, by which it is possible to remove personal information and information allowing the person to be inferred from information unit data encompassed by the personal data, or to convert the personal information and information allowing the person to be inferred automatically or manually by the user.

23. The data network of claim 22, wherein the computer unit associated with the user comprises a computer, a tablet, a cell phone, a smart phone, a smart watch, a wearable or a PDA.

24. The data network of claim 22, wherein the computer unit associated with the user having the control logic includes a recognition logic, by which it is possible to automatically recognize personal information and information allowing the person to be inferred in the information unit data and to remove it from the information unit data.

25. The data network of any one of claim 22, wherein the computer unit associated with the user having the control logic converts personal information and information allowing the person to be inferred into generalized personal information and information allowing the person to be inferred.

26. The personal data network of claim 24, wherein:
the recognition logic comprises a text, image or audio recognition logic;
the control logic, by way of a comparison of recognized words, images or audio components to predetermined words, images, audio components, or formation laws, identifies personal information and information allowing the person to be inferred; and
the control logic removes the identified personal information from the information unit data or converts it into generalized personal information and information allowing the person to be inferred.

27. The personal data network of claim 22, wherein the computer unit associated with the user having the control logic by which information unit data is output on an output of the computer unit, and that enables the user to remove personal information and information allowing the person to be inferred, identified in the information unit data based on the output, or to convert it into generalized personal information and information allowing the person to be inferred.

28. The personal data network of claim 22, wherein the computer unit associated with the user having the control logic by which personal information and information allowing the person to be inferred that was removed from the information unit data, or converted into generalized personal information and information allowing the person to be inferred, is stored, and which from the de-personalized personal data, which was received from the server device via the network and in which personal information and information allowing the person to be inferred was removed from information unit data, or converted into generalized personal information and information allowing the person to be inferred, and the stored personal information at least partially reconstructs the original information unit data including the personal information.

29. The personal data network of claim 22, wherein the computer unit associated with the user having the control logic enables a transmission of personalized personal data or a transmission of the allocation rule to a supporter computer device.

30. The personal data network of claim 29, further comprising an analysis interface of the personal data network connected to an analysis computer device.

31. The personal data network of claim 30, wherein the supporter computer device, the analysis computer device, the server device or the computer unit associated with the user having the control logic ascertains findings or generates automatic messages from the de-personalized personal data of a user.

32. The personal data network of claim 30, wherein the supporter computer device, the analysis computer device, the server device or the computer unit associated with the user have an acquisition device by which personalized or de-personalized personal data can be acquired.

33. The personal data network of claim 22, further comprising a transmission path disconnection device, interposed between the computer unit associated with the user and the server device, that transmits the de-personalized personal data transmitted from the computer unit to the server device and makes it impossible to infer the transmission path from the computer unit and from de-personalized personal data on the server device.

34. The personal data network of claim 22, wherein, for a registration of the user, the computer unit associated with the user has a control logic that:
sends a telephone number of the computer unit;
receives a code for authenticating the computer unit; and
records personal data of the user, which is then stored in the computer unit, only after the code for authenticating the computer unit has been received.

35. The personal data network of claim 22, wherein the computer unit associated with the user comprises a control logic that:
after transmitting the de-personalized personal data from the server device to the computer unit or the supporter computer device, or after transmission of the allocation rule or identifier to a supporter computer device, ascertains a new allocation rule with a new identifier, and
transmits the de-personalized personal data with an identifier from the computer unit to the server device via the network.

36. The data network of any one of claim 22, wherein the allocation rule is transmitted to a person of a trust computer unit associated with a person of trust, and it is made possible for the person of the trust computer unit to at least partially access the de-personalized personal data and convert it into personalized personal data.

37. The personal data network of any one of claim 22, wherein the de-personalized personal data, with respect to the user, includes:
a year of birth;
a gender;
an ethnicity;
a race; or
a country of a place of residence.

38. The personal data network of any one of claim 22, wherein the computer unit comprises a portable computer unit.

39. The personal data network of claim 22, wherein a control logic of the server device is designed to generate automatic messages to the user or another predetermined person from the transmitted de-personalized data.

40. The personal data network of claim 22 further comprising a vital data acquisition device.

41. The personal data network of claim 40, wherein the vital data acquisition device comprises one or more sensors for acquiring vital data.

42. The personal data network of claim 41, wherein the one or more sensors comprise a heart rate sensor, a blood pressure sensor, a glucose level sensor, a cardiac pacemaker, a fitness tracker, a position sensor, or an acceleration sensor.
